(19) United States
(12) Patent Application Publication
(10) Pub. No.: US 2007/0033749 A1
(43) Pub. Date: Feb. 15, 2007

(54) Anionic azo dyes and the metal complexes thereof ofr colouring leather

(75) Inventors: Laszlo Somogyi, LIMBURGERHOF (DE); Andrea Zamponi, Mannheim (DE)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 10/559,333

(22) PCT Filed: Jun. 4, 2004
(86) PCT No.: PCT/EP04/06045

§ 371 Date: Dec. 5, 2005

(30) Foreign Application Priority Data
Jun. 6, 2003 (DE) 103 25 808.6

(51) Int. Cl. C09B 29/08; C09B 33/18; C07C 245/02

(52) U.S. Cl. 8/662; 534/805

(57) ABSTRACT

The present invention relates to the use of anionic azo dyes which have at least one acidic group in the form of its salt and at least 2 azo groups and also to the use of their anionic metal complexes for dyeing leather.

The present invention relates to the use of anionic azo dyes which have at least one acidic group in the form of its salt and at least 2 azo groups and also to the use of their anionic metal complexes for dyeing leather.

Anionic azo dyes and their use for dyeing leather are generally known, for example from G. Otto “Das Färben von Leder”, Eduard Roether Verlag, Darmstadt 1962, Heinrich Zollinger “Color Chemistry, Synthesis, Properties and Applications of Organic Dyes and Pigments”, 2nd Ed. VCH Weinheim 1991, and also from DE-A 3212240, DE-A 3124172, EP-A 34724, EP-A 78007, EP-A 598244, EP-A 692523, EP-A 577556, EP-A 421927, EP-A 322357, EP-A 316278 and also from “Bibliothek des Leders” volume 5 (the dyeing of leather) [1987] Umschau Verlag. Typically, such dyes are used in the form of their salts or in the form of salts of anionic transition metal complexes of such azo dyes, especially in the form of complexes of iron, of cobalt, of nickel, of copper, of manganese, of zinc, of cadmium and/or of chromium. Such salts typically contain sodium as counterions.

There have been various reports about aqueous dye formulations of anionic azo dyes and also about aqueous formulations of anionic metal complexes of azo dyes that contain lithium ions or organic ammonium ions as counterions. These counterions improve the solubility of the azo dyes and thus make it possible to provide more concentrated dye formulations (see for example DE-A 2405855, DE-A 2443483, DE-A 3322502, DE-A 3529495).

Of particular interest for leather dyeing are anionic azo dyes and anionic transition metal complexes of azo dyes whose dye anion is described by the general formula I: 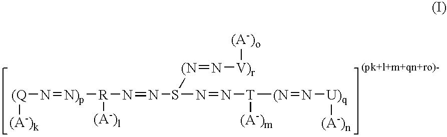
where
Q, R, T, U and V are independently a benzene, naphthalene, diphenylamine, diphenylmethane, phenylaminosulfonylbenzene or phenylaminocarbonylbenzene radical (although Q and U may also be a pyrazole, triazole, isothiazole or thiadiazole and thiazole radical) and as well as by the optionally present anionic group A⁻ may optionally be substituted by one or more of the following radicals: OH, NH₂, NO₂, halogen, C₁-C₄-alkyl, C₁-C₄-hydroxyalkyl, C₁-C₄-alkoxy, C₁-C₄-alkylamino, C₁-C₄-dialkylamino, C₁-C₄-alkylaminocarbonyl, C₁-C₄-dialkylaminocarbonyl, C₁-C₄-alkylcarbonylamino, N-(C₁-C₄-alkylcarbonyl)-N-(C₁-C₄-alkylcarbonyl)amino, phenylcarbonylamino, formamide or N-attached 5- or 6-membered heterocyclyl, although Q and U may independently have an —N═N—P substituent, where P may be an aromatic radical which is selected from the group consisting of phenyl, naphthyl, pyridyl, 2-pyridonyl, pyrazolyl and thiazolyl and which may optionally be substituted by one or more of the following radicals: OH, NH₂, NO₂, halogen, C₁-C₄-alkyl, C₁-C₄-hydroxyalkyl, C₁-C₄-alkoxy, C₁-C₄-alkylamino, C₁-C₄-dialkylamino, C₁-C₄-alkylaminocarbonyl, C₁-C₄-dialkylaminocarbonyl, C₁-C₄-alkylcarbonylamino, N-(C₁-C₄-alkylcarbonyl)-N-(C₁-C₄-alkylcarbonyl)amino, phenylcarbonylamino, formamide or N-attached 5- or 6-membered saturated nitrogen heterocyclyl, for example morpholinyl, piperazinyl or pyrrolidinyl;
S is a radical of the formulae A or B: 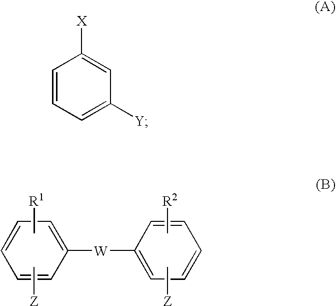
where X and Y in the formula A are independently OH, NH₂, C₁-C₄-alkyl, C₁-C₄-hydroxyalkyl, C₁-C₄-alkoxy, C₁-C₄-alkylamino, C₁-C₄-dialkylamino or N-attached 5- or 6-membered saturated heterocyclyl, W in the formula B is a bond, O, NH, SO₂, SO₂NH, CONH or C₁-C₄-alkylene, R¹ and R² are independently hydrogen, OH, or C₁-C₄-alkyl and each Z is the same or different and represents COOH or hydrogen;
A⁻ may at each instance be the same or different and is selected from the group consisting of CO₂⁻ and SO₃⁻;
k, l, m, n and o may be the same or different and are each 0, 1 or 2, provided the sum total pk+l+m+qn+ro is at least 1 and especially at least 2, for example 2, 3, 4, 5 or 6;
p, q and r are independently 0 or 1, and the sum total p+q+r is preferably 1 or 2.

Such anionic azo dyes are known from the above-cited prior art and for example also as acid dyes from the Colour Index, Database Producer, Society of Dyers and Colourists and American Association of Textile Chemists and Colourists, Datastock inclusive July 1999, and have variously been proposed for leather dyeing.

Anionic azo dyes whose dye anions are described by the formula I and also anionic transition metal complexes thereof generally contain sodium as counterions. However, the migration fastness of such dyes and of the corresponding transition metal complexes leaves something to be desired.

It has now been found that, surprisingly, such dyes and dye transition metal complexes exhibit improved migration fastness when the cations required to neutralize the dye anion I or the transition metal complex of I comprise lithium ions and sodium ions in a molar ratio in the range from 1:19 to 4:1 and especially in a molar ratio in the range from 1:9 to 3.5:1.

The present invention thus provides for the use of anionic azo dyes which have at least one acidic group in the form of its salt and at least 2 azo groups and whose dye anion is described by the general formula I that comprise lithium ions and sodium ions in a molar ratio in the range from 1:19 to 4:1 and especially in a molar ratio in the range from 1:9 to 3.5:1, for dyeing leather.

The molar ratio of Na to Li ions which is to be observed according to the present invention always relates to the total amount of free cations present in the dye. The aforementioned lithium ions and sodium ions typically account for at least 90% and at least 95% of the free, ie uncomplexed, cations present in the dye. The uncomplexed cations serve to neutralize the anionic groups in the dye anion I and also to neutralize the inorganic cations such as chloride, nitrate or sulfate which are possibly present in the dye owing to the nature of the preparative process.

Preferably, the dye comprises less than 10 mol % and especially less than 5 mol % of counterions other than lithium and sodium, based on the total amount of free, ie uncomplexed, cations present in the dye. It will be appreciated that the dye, as well as the dye anion I, can additionally comprise further anions; these are generally, owing to the nature of the preparative process, inorganic anions of inorganic acids, examples being chloride, nitrate, sulfate and the like. The proportion of cations required to neutralize the anions is typically not more than 70 mol % especially not more than 50 mol % of the free cations present in the dye.

It will be appreciated that the maximum charge of the dye anion is equal to the number of anionic groups A⁻ in the dye anion. The dye anion, however, can also be present in the dye in partly neutralized form, in which case it is customary for the dye anion in the dye to typically have a net charge of at least 1.

Also suitable for use according to the present invention are transition metal complexes of the aforementioned anionic azo dyes, especially complexes of the transition metals of the groups VI to X of the periodic table of the elements and of these especially complexes of Cu, Cr, Fe, Ni, Co, Mn, Zn and Cd. The molar ratio of transition metal to dye anion in these metal complexes corresponds to the stoichiometry typical for the complex and is customarily in the range from 2:1 to 1:2. Since, in general, transition metal ions are complexed not via the anionic groups A⁻ but via deprotonated hydroxyl groups and amino groups, the positive charge of the metal cation customarily makes no or only a partial contribution to charge-neutralized in the dye anion 1, so that the metal complexes have a negative overall charge which is neutralized by the alkali metal ions present in the dye.

Also suitable are obviously mixtures of such azo dyes and also mixtures of such azo dyes with their transition metal complexes and mixtures of the complexes with each other.

The naphthalene radicals mentioned in connection with the groups Q-(A⁻)ₖ, R-(A⁻)ₗ, T-(A⁻)ₘ, U-(A⁻)ₙ or V-(A⁻)ₒ represent for example a radical of the formula II: 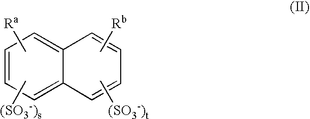
where R^{a} and R^{b} are independently hydrogen or one of the radicals mentioned above as substituents and especially hydrogen, OH, NH₂ or NHC(O)R³, where R³ is hydrogen, C₁-C₄-alkyl or phenyl. In the formula II s and t are each 0 or 1 provided the sum total s+t is 1 or 2. Examples of radicals of the formula II are those which are derived from sulfonated α-naphthols such as 4-hydroxysulfonyl-α-naphthol, azuric acid (5-hydroxysulfonyl-α-naphthol), epsilon acid (3,8-bis(hydroxysulfonyl)-α-naphthol), 3,6-bis(hydroxysulfonyl)-α-naphthol, 4,8-bis(hydroxysulfonyl)-α-naphthol, 3,6-bis(hydroxysulfonyl)-8-hydroxy-α-naphthol, sulfonated β-naphthols such as 6-hydroxysulfonyl-β-naphthol, 8-hydroxysulfonyl-β-naphthol, 3,6-bis(hydroxysulfonyl)-β-naphthol, 3,8-bis(hydroxysulfonyl)-β-naphthol, sulfonated β-naphthylamines such as 5-aminonaphthalene-1-sulfonic acid, 5-aminonaphthalene-2-sulfonic acid (=naphthylaminesulfonic acid-1,6=Cleve's acid 6), naphthylaminesulfonic acid-1,7 (=Cleve's acid 7), 4-aminonaphthalene-1-sulfonic acid, sulfonated β-naphthylamines such as 2-aminonaphthalene-1-sulfonic acid, 2-aminonaphthalene-5-sulfonic acid, 2-aminonaphthalene-6-sulfonic acid, 2-aminonaphthalene-4,8-disulfonic acid, 2-aminonaphthalene-5,7-disulfonic acid, 2-aminonaphthalene-3,6-disulfonic acid, sulfonated aminohydroxynaphthalenes and acylated derivatives such as H acid (=8-amino-3,6-bishydroxysulfonyl-α-naphthol), acet-H acid (=8-acetylamino-3,6-bishydroxysulfonyl-α-naphthol), benzoyl-H acid (=8-benzoylamino-3,6-bishydroxysulfonyl-α-naphthol), K acid, (=8-amino-3,5-bishydroxysulfonyl-α-naphthol), acet-K acid (=8-acetylamino-3,5-bishydroxysulfonyl-α-naphthol), benzoyl-K acid (=8-benzoylamino-3,5-bishydroxysulfonyl-α-naphthol), 7-amino-3-hydroxysulfonyl-α-naphthol, 8-amino-5-hydroxysulfonyl-α-naphthol, 8-amino-5,7-bishydroxysulfonyl-α-naphthol, J acid (=6-amino-3-hydroxysulfonyl-α-naphthol), methyl-J acid (=6-methylamino-3-hydroxysulfonyl-α-naphthol) or M acid (=5-amino-3-hydroxysulfonyl-α-naphthol), especially from H acid, acet-H acid, benzoyl-H acid, K acid, J acid, purpurin.

The diphenylamine, phenylaminosulfonylbenzene and phenylaminocarbonylbenzene radicals mentioned in connection with the groups Q-(A⁻)ₖ, R-(A⁻)ₗ, T-(A⁻)ₘ, U-(A⁻)ₙ or V-(A⁻)ₒ represent for example a radical of the formula III: 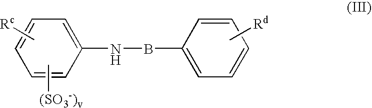
where R^{c} and R^{d} are independently hydrogen or one of the radicals mentioned as substituents. When B is a single bond, v is preferably 1. When B is a CO or SO₂ group, v is preferably 0. R^{c} and R^{d} are each then in particular hydrogen. Examples of radicals of the formula III are those which are derived from 2-(4-aminophenyl)amino-5-nitrobenzenesulfonic acid or from phenylaminosulfonylbenzene.

The diphenylmethane radicals mentioned in connection with the groups Q-(A⁻)ₖ, R-(A⁻)ₗ, T-(A⁻)ₘ, U-(A⁻)ₙ or V-(A⁻)ₒ represent for example a radical of the formula IVa: 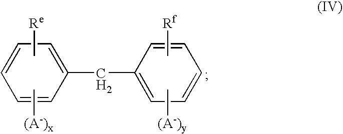
where A⁻ is as defined above, R^{e} and R^{f} are independently hydrogen or one of the radicals mentioned as substituents and especially hydrogen, OH or C₁-C₄-alkyl, and x and y are independently 0 or 1. Examples thereof are the radicals derived from 2,2′-dihydroxy-3,3′-dicarboxyldiphenylmethane; 2,2′-dimethyidiphenylmethane and 2,2′-dicarboxyldiphenylmethane.

The phenyl radicals mentioned in connection with the groups Q-(A⁻)ₖ, R-(A⁻)ₗ, T-(A⁻)ₘ, U-(A⁻), or V-(A⁻)ₒ represent for example a radical of the formula V: 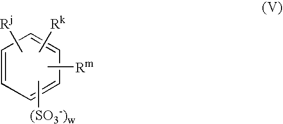
where Rⁱ, R^{k} and Rₘ are independently hydrogen or one of the radicals mentioned as substituents and w is 0, 1 or 2. When w is 0, especially one of Rⁱ, Rₖ and Rₘ is other than hydrogen. Preferred radicals other than hydrogen are: nitro, OH, C₁-C₄-alkoxy, C₁-C₄-alkyl, halogen, amino, C₁-C₄-alkylamino, di-C₁-C₄-alkylamino and C₁-C₄-alkylcarbonylamino. Examples of preferred radicals of the formula V are those which are derived from benzenesulfonic acid, 1,3-benzenedisulfonic acid, 2-, 3- or 4-nitrobenzenesulfonic acid, nitrobenzene, phenol, 2-, 3- or 4-nitrophenol, 2,4-dinitrophenol, 4-nitrophenol-6-sulfonic acid, resorcinol, toluene, aniline, anisole, benzoic acid, m- or p-diaminobenzene, metamine or metaminic acid.

Examples of radicals which are derived from pyrazole, thiazole, isothiazole, triazole or thiodiazole comprise radicals derived from 3-methylpyrazol-5-one, 1-phenyl-3-methylpyrazol-5-one, 1-(4-hydroxysulfonylphenyl)-3-methylpyrazol-5-one, 1-(4-hydroxysulfonyl-2-methylphenyl)-3-methylpyrazol-5-one, 1-(4-hydroxysulfonylphenyl)-3-carboxylpyrazol-5-one, 1-(2,5-bishydroxysulfonylphenyl)-3-methylpyrazol-5-one, triazolecarboxylic acid, benzothiazole, indazole, 5-nitrobenzisothiazole, 5-nitro-7-bromo-2,1-benzisothiazole or 5-phenylthiodiazole.

A preferred embodiment of the invention is based on the use of anionic azo dyes and of metal complexes thereof in which S in the dye anion of the formula I represents a radical of the formula A where X and Y are each as defined above and are each in particular OH.

In a further preferred embodiment of the invention S represents a radical of the formula B.1, B.2, B.3 or B.4: 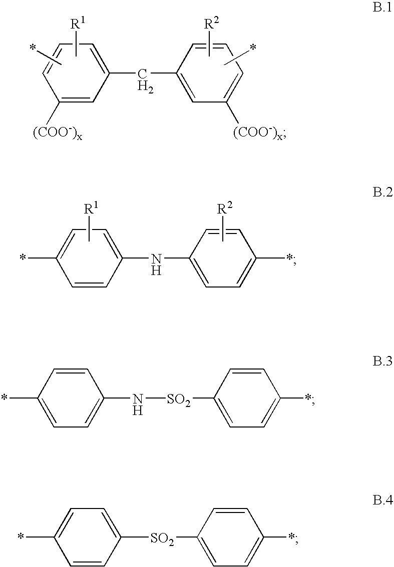
where * denotes the bonds to the azo groups and R¹, R² in the formulae B.1 and B.2 are each as defined above and are in particular independently H, C₁-C₄-alkyl or OH.

In a preferred embodiment of the invention, at least one of the groups Q-(A⁻)ₖ, R-(A⁻)ₗ, T-(A⁻)ₘ, U-(A⁻)ₙ or V-(A⁻)ₒ or especially one or two of these groups is described by the above-described general formula II: 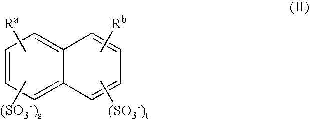
where R^{a} and R^{b} are independently hydrogen, OH, NH₂ or NHC(O)R³, where R³ is hydrogen, C₁-C₄-alkyl or phenyl, s and t are each 0 or 1 and the sum total s+t is 1 or 2. In particular, the R-(A⁻)ₗ group then represents a radical of the formula II. Particular preference is therefore given to dyes and complexes thereof wherein the dye anion has the formula Ia: 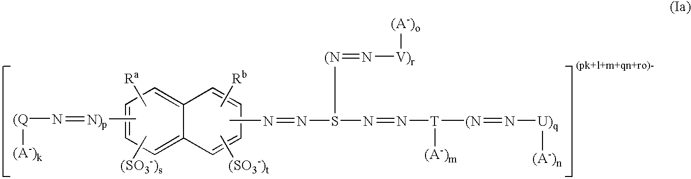
where R^{a}, R^{b}, A⁻, S, T, U, V, k, m, n, p, q, o, r are each as defined above, a and b are independently 0 or 1, s and t are independently 0 or 1 and the sum total s+t is 1 or 2. Preferably, r in the formula la is 0. Preferably, the sum total p+q+r is 0 or 1 and especially 1.

In the formulae I and Ia T-(A⁻)ₘ preferably represents a radical of the formulae III or V. In the formulae I and Ia Q-(A⁻)ₖ preferably represents a radical of the formulae III or V when p=1. U-(A⁻)ₙ preferably represents a radical of the formula V.

Examples of azo dyes which are particularly preferred according to the present invention are those in which the anions I and la have the following formulae Ia.1, Ia.2, Ia.3, Ia.4, Ia.5, Ia.6 or Ia.7:

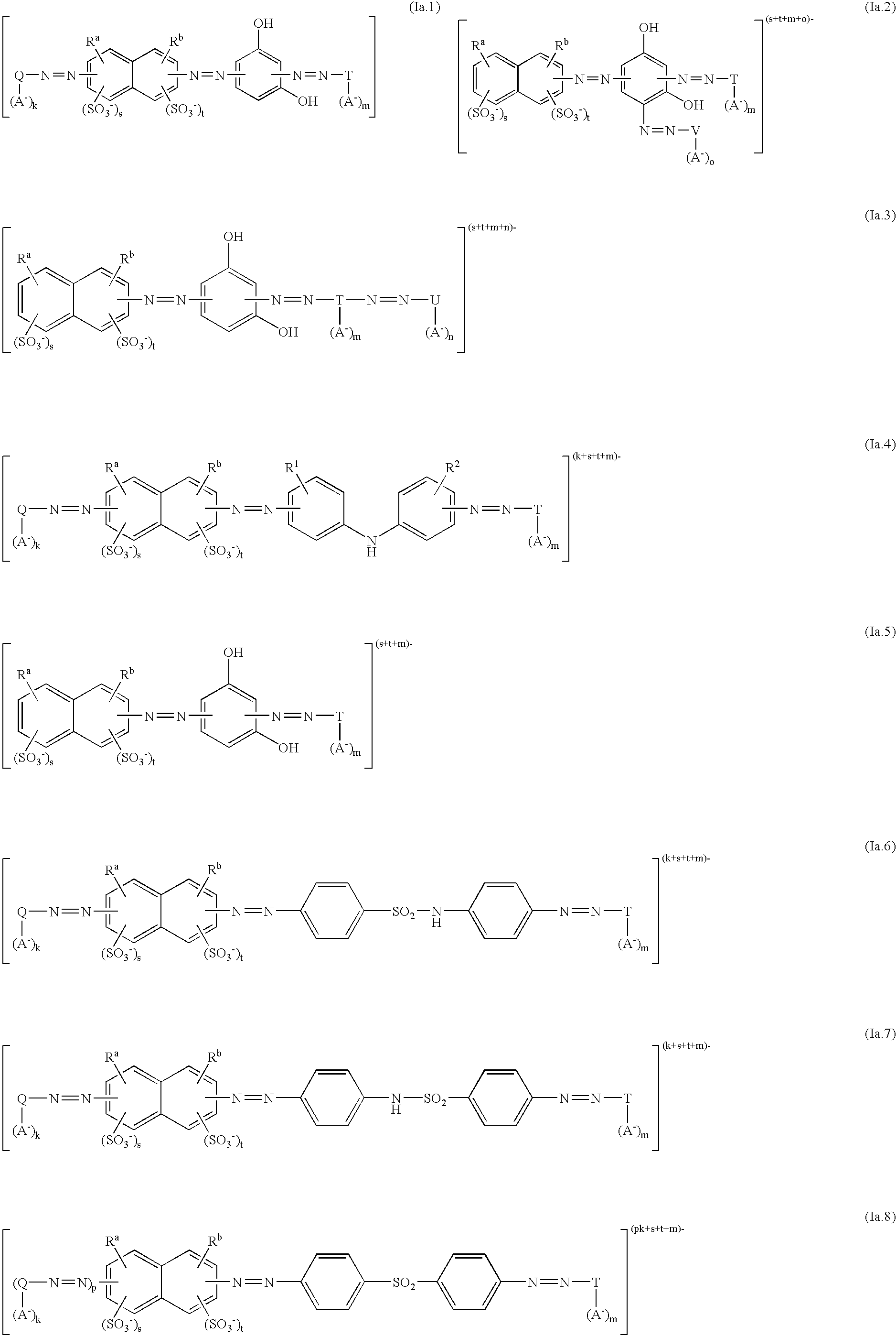

In the formulae Ia.1 to Ia.8, the variables R^{a}, R^{b}, R¹, R², A⁻, T, U, V, k, m, n, o and p are each as defined above. The variables s and t are independently 0 or 1 subject to the proviso that the sum total s+t is 1 or 2. R¹ and R² in the formula Ia.4 are each preferably H or OH.

In the formulae Ia.1 to Ia.8 T-(A⁻)ₘ is preferably a radical of the formulae III or V. In the formulae Ia.1, Ia.4 and Ia.6 Q-(A⁻)ₖ is preferably a radical of the formulae III or V and especially a radical of the formula V. In the formula Ia.6 Q-(A⁻)ₖ is especially a radical of the formula V. In the formula Ia.6 T-(A⁻)ₘ is in particular a radical of the formula V. In the formulae Ia.7 and Ia.8 T-(A⁻)ₘ is especially a radical of the formula II. The variable p is especially 0. In the case p≠0 Q-(A⁻)ₖ is especially a radical of the formula V.

Of the compounds of the formulae I and Ia preference is especially given also to those compounds in which at least one of the groups Q-(A⁻)ₖ, R-(A⁻)ₜ, T-(A⁻)ₘ, U-(A⁻)ₙ or V-(A⁻)ₒ is described by the general formulae IIIa: 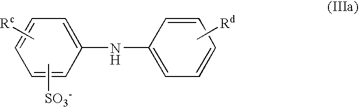
where R_{c} and R_{d} are independently hydrogen, OH, NH₂, NO₂, halogen, C₁-C₄-alkyl, C₁-C₄-hydroxyalkyl, C₁-C₄-alkoxy, C₁-C₄-alkylamino, C₁-C₄-dialkylamino, C₁-C₄-alkylaminocarbonyl, C₁-C₄-dialkylaminocarbonyl, C₁-C₄-alkylcarbonylamino, N-(C₁-C₄-alkylcarbonyl)-N-(C₁-C₄-alkylcarbonyl)amino or N-attached 5- or 6-membered saturated nitrogen heterocyclyl. When R-(A⁻)ₗ or T-(A⁻)ₘ represent a radical of the formula IIIa, they preferably do not contain any further radical Q-(A⁻)ₖ or U-(A⁻)ₙ respectively attached via an azo group. In the formula IIIa R^{d} is especially hydrogen. R^{c} is especially NO₂.

In a further preferred embodiment of the invention S in the formula I is a radical of the formula b.1. Preference thereamong is given to dyes of the hereinbelow indicated general formula Ib where the groups Q-(A⁻)ₖ, R-(A⁻)ₗ, T-(A⁻)ₘ and U-(A)ₙ are each as defined above. In particular, for p and/or q the groups Q-(A⁻)ₖ and U-(A⁻)ₙ are derived from radicals of the general formula V.

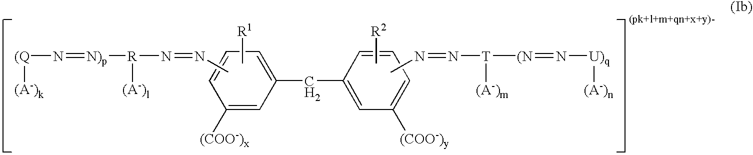

In the formula Ib, A⁻, Q, R, R¹, R², T, U, k, l, m, n, p and q are each as defined above. The variables x and y are independently 0 or 1. When p and/or q are not equal to 0, Q-(A⁻)ₖ and U-(A)ₙ are each preferably a benzene radical of the formula V. R-(A⁻)ₗ and T-(A⁻)ₘ are each preferably a benzene radical of the formula V, a radical of the formula IIIa or a naphthalene radical of the formula II.

Examples of azo dyes of the formula lI are the dyes of the general formulae Ib.1, Ib.2 and Ib.3:

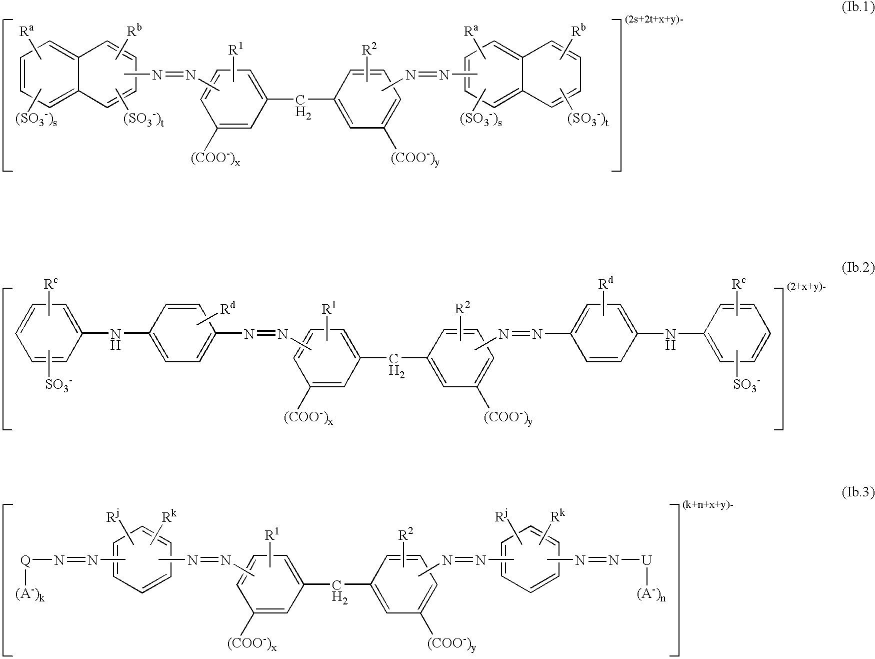

In the formula Ib.1 to Ib.3, m, n, s, t, x, y, A⁻, Q, U, R¹, R², R^{c}, R^{d}, Rⁱ and R^{k} are each as defined above. In the formula Ib.3, k and n are each especially 1. A⁻ is especially SO₃⁻. Q-(A⁻)ₖ and U-(A⁻)ₙ are each in particular a radical of the formula V where w=1.

Preference is further given to the use of azo dyes and their metal complexes in which S is a radical of the formula A and the groups Q-(A⁻)ₖ, R-(A⁻)ₗ, T-(A⁻)ₘ, U-(A⁻)ₙ and V-(A⁻)ₒ represent radicals of the formula III or V. These include in particular compounds in which p and/or q are each 0 or 1 and especially 0. In these dyes, the anions of the formula I can be represented especially by the formula Ic: 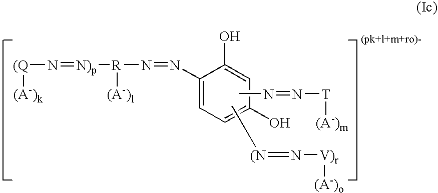
where A⁻, k, l, m, o, p and r are each as defined above, R and T are independently a benzene or diphenylamine radical, V and Q are independently a benzene radical and R, T, Q and V as well as the anionic group A⁻ optionally present may optionally be substituted by one or more of the following radicals: OH, NH₂, NO₂, halogen, C₁-C₄-alkyl, C₁-C₄-hydroxyalkyl, C₁-C₄-alkoxy, C₁-C₄-alkylamino, C₁-C₄-dialkylamino, C₁-C₄-alkylaminocarbonyl, C₁-C₄-dialkylaminocarbonyl, C₁-C₄-alkylcarbonylamino, N-(C₁-C₄-alkylcarbonyl)-N-(C₁-C₄-alkylcarbonyl)amino. R-(A⁻)ₗ, T-(A⁻)ₘ represent in particular radicals of the formula III, specifically IIIa or are each a radical of the formula V. For p=1, Q-(A⁻)ₖ is in particular a radical of the formula V. For r=1, V-(A⁻)ₒ is in particular a radical of the formula V. p+r is in particular 0 or 1.

The dyes and their metal complexes to be used according to the present invention are prepared in a conventional manner by a multistep diazotization/coupling sequence which comprises at least two successive azo couplings, of which at least the last one is carried out in an aqueous reaction medium which comprises at least 90 mol %, based on the metal ions comprised in the reaction medium, of sodium ions and lithium ions in a Na:Li molar ratio in the range from 95:5 to 20:80.

In other words, the process for preparing the dye anions I comprises:

i) a first step of diazotizing a first aromatic amine or tetrazotizing an aromatic diamine and coupling the resultant diazonium compound onto 1 mol equivalent of a coupling component or the tetrazonium compound with from 1 to 2 mol equivalents of a coupling component to obtain an azo dye or a disazo dye, and

ii) a second step of coupling at least one further diazotized amine onto the dye obtained in the first step.

Step ii) may be followed by further azo couplings, for example by coupling diazonium compounds onto the dye obtained in step ii) or by diazotizing a primary amino group present in the dye and coupling the resultant diazonium compound onto a coupling component.

Mol equivalents here and below refers to the stoichiometry of the reaction. However, individual reactants can be used not only in excess but also in deficiency, based on the desired stoichiometry. The departure from the desired stoichiometry will generally be not more than 20 mol % and especially not more than 10 mol %. In other words, when a diazonium component is reacted with a coupling component, the molar ratio of the two components will be in the range from 1:1.2 to 1.2:1 and especially in the range from 1.1:1 to 1:1.1. When a tetrazonium compound is reacted with 2 mol equivalents of a coupling component, the coupling component will accordingly be used in an amount from 1.6 to 2.4 mol and especially in an amount from 1.8 to 2.2 mol per mole of tetrazonium compound.

The reaction conditions required for the diazotization and coupling are well known to one skilled in the art, for example from the prior art cited at the beginning. They typically take place in an aqueous reaction medium.

The diazotization and coupling of the resultant diazonium compound or of the tetrazonium compound typically takes place in an aqueous reaction medium under pH control in a conventional manner. Preferably, the lithium ions are introduced into the dye in the form of lithium bases such as lithium hydroxide or lithium carbonate for pH adjustment before the coupling and/or for pH control during the coupling. More particularly, the molar ratio of sodium ions to lithium ions is during at least one coupling step in the range from 95:5 to 20:80 and especially in the range from 1:9 to 7:3. Preferably, less than 10 mol % and especially less than 5 mol % of the uncomplexed metal cations present in the reaction mixture differ from sodium and lithium ions.

Following the diazotization/coupling sequence, the transition metal complexes may be prepared by adding the desired transition metal salt in the form of a suitable, preferably water-soluble salt in the stoichiometrically desired amount and if necessary heating to the temperature required for complexing, for example at temperatures in the range from 40 to 100° C.

Particular preference is given to using dyes in which the dye anion of the formula I is obtainable by a process which comprises the following steps:
i) coupling a first diazonium compound D1 which has at least one anionic group A⁻ and which has a diazonium group onto a coupling component of the general formulae A or B in an aqueous reaction medium to obtain an azo dye A1 which preferably has at least one anionic group and at least one azo group, and
ii) coupling a second di- or tetrazonium component D2 which optionally has one or more anionic groups A⁻ and one or two diazonium groups onto the azo dye A1 in the aqueous reaction mixture obtained in step i), and optionally
iii) coupling at least one third diazonium compound D3 which optionally has one or more anionic groups A⁻ at least one diazonium group onto the azo dye obtained in step ii) in the aqueous reaction mixture obtained in step ii) or, when a tetrazonium component was used in step ii), coupling a coupling component onto the azo dye obtained in step ii),
   wherein the aqueous reaction mixture at least in the last of the coupling steps ii) or iii) comprises lithium ions in an amount from 5 to 80 mol % and especially 10 to 70 mol %, based on the total amount of the uncomplexed metal cations present in the reaction mixture.

This process is especially useful for preparing azo dyes wherein the group S represents a group of the formula A or B.2. The diazonium components D1 then correspond to the groups R and T respectively and the diazonium component D3 to the groups T, R, U and Q. In other words, the diazonium compound D1 comprises compounds of the general formula (A⁻)ₗ-R-N₂⁺, (A⁻)ₘ-T-N₂₊, (A⁻)ₖ-Q-N═N-[(A⁻)ₗ-R]-N₂⁺ or (A⁻)ₙ-U-N═N-[(A⁻)ₘ-T]-N₂⁺, where k, l, m, n, A, Q, R, T and U are each as defined above. The diazonium component D2 correspondingly comprises (A⁻)ₗ-R-N₂⁺, (A⁻)ₘ-T-N₂⁺, (A⁻)ₖ-Q-N₂⁺ or (A⁻)ₙ-U-N₂⁺ and the tetrazonium component D2 comprises ⁺N₂-[(A⁻)ₗ-R]-N₂₊ or ⁺N₂-[(A⁻)ₘ-T]-N₂⁺. The diazonium component D3 correspondingly comprises (A⁻)ₒ-V-N₂₊, (A⁻)ₖ-Q-N₂⁺ or (A⁻)ₙ-U-N₂⁺.

Particular preference is further given to using dyes wherein the dye anion of the formula I is obtainable by a process which comprises the following steps:
i) coupling a tetrazonium compound Ti which may optionally have one or two anionic groups A⁻ and which has two diazonium groups onto a coupling component K1 in an aqueous reaction medium which comprises alkali metal cations to obtain an azo dye A2 which optionally has one or more acidic groups, an azo group and a diazonium group or which has two azo groups, and
ii) coupling a second azonium component D4 which optionally has one or more anionic groups A⁻ and at least one diazonium group onto the azo dye A2 in the aqueous reaction mixture obtained in step i) or coupling the diazonium compound obtained in step i) onto 1 mol equivalent of a coupling component K2, and optionally
iii) coupling a third diazonium component D5 which optionally has one or more anionic groups A⁻and a diazonium group onto the azo dye obtained in step ii) in the aqueous reaction mixture obtained in step ii) or coupling the diazonium compound obtained in step ii) onto 1 mol equivalent of a coupling component 3,
   wherein the aqueous reaction mixture at least in the last of the coupling steps ii) or iii) comprises lithium ions in an amount from 5 to 80 mol % and especially 10 to 70 mol %, based on the total amount of the uncomplexed cations present in the reaction mixture.

This process is especially useful for preparing azo dyes wherein the group S represents a group of the formula B.1, B.3 or B.4. The tetrazonium component T1 corresponds to the group S, the coupling component to the groups R and T and the diazonium component D4 and D5 to the groups Q, V and U. In other words, the tetrazonium compound has a general formula ⁺N₂-S-N₂⁺, where S represents a radical of the formulae B.1, B.3 or B.4. The coupling component K1 conforms to the formulae (A⁻)ₗ-R, (A⁻)ₘ-T, (A⁻)ₖ-Q-N═N-R-(A⁻)I or (A⁻)ₙ-U-N═N-T-(A⁻)ₘ. The diazonium component D4 correspondingly comprises (A⁻)ₗ-R-N₂⁺, (A⁻)ₘ-T-N₂⁺, (A⁻)ₖ-Q-N₂⁺ or (A⁻)ₙ-U-N₂⁺ and the coupling component K2 correspondingly comprises compounds of the type (A⁻)ₗ-R, (A⁻)ₘ-T, (A⁻)ₖ-Q or (A⁻)ₙ-U. Diazonium components D5 correspondingly represent (A⁻)ₖ-Q-N₂⁺, (A⁻)ₙ-U-N₂⁺ or (A⁻)ₒ-V-N₂⁺.

The dye is isolated from the aqueous reaction mixture in a conventional manner, for example by evaporating and especially by spray-drying the aqueous reaction mixture, by salting out the dye and drying the presscake. To prepare a liquid brand or formulation of the dye, the dye presscake, or the dye powder obtained by evaporation or spray drying, can be dissolved. It is also possible to prepare the liquid formulation of the dye directly from the reaction solution or by ultrafiltration and spray drying. It may then be necessary for a sufficiently high dye concentration and for the stability of the liquid brand to carry out a dia- or ultrafiltration in order that the inorganic salts formed in the course of the synthesis may be depleted and the solution concentrated.

The dyes to be used according to the present invention may in principle be applied to all types of leather, ie intermediate articles which have not been retanned, such as metal oxide tanned leather and organic or vegetable-tanned leather, and also intermediate articles which have been retanned, such as bark or crust leather.

The present invention thus also provides a process for dyeing leather, which comprises treating leather with a dye-comprising aqueous liquor which comprises at least one anionic azo dye or an anionic transition metal complex of an anionic azo dye of the kind described herein.

Leather dyeing including retanning, fatliquoring and aftertreatment is effected in a conventional manner, for example by dyeing in a drum, in a paddle, by brush dyeing or by spray dyeing. Such processes are extensively described in the prior art, for example in: “Bibliothek des Leders”, Volume 3 (tanning agents, tanning and retanning) [1985], Volume 4 (defatting, fatliquoring and hydrophobicization in leather manufacture) [1987] & Volume 5 (the dyeing of leather) [1987] Umschau Verlag; “Leather Technicians Handbook”, 1983, by J. H. Sharphouse, published by Leather Producers Association; and “Fundamentals of Leather Manufacturing”, 1993, by E. Heidenmann, published by Eduard Roether KG.

Dyeing can be carried out not only after tanning, ie during retanning, but also after retanning, not only in the same but also in a separate bath. Dyeing is preferably carried out during retanning. The dyeing operation is generally followed by a fatliquoring operation. Retanning, dyeing and fatliquoring can also be carried out in one process step, however. In general, not only the dyeing but also the fatliquoring and the retanning will be fixed at the end of the operation by acidification, ie a final acidification takes place after dyeing and optionally retanning and fatliquoring. Typically, for acidification, the pH of the aqueous treatment bath is adjusted to a value above 3.5 by addition of an acid, especially formic acid. Fixation occurs in the process. The degree of fixation can be improved by using fixing assistants which are customary for this purpose, which are typically cationic polymers.

The dyeing is typically carried out in an aqueous liquor. The dye is customarily used for this in an amount from 1 to 12% by weight, based on the shaved weight of the leather used. The liquor may comprise customary anionic dyeing assistants, nonionic surface-active substances and also tanning materials customarily used for retanning, for example polymeric retanning materials, vegetable tanning materials and fatliquors.

The dyeing with the dyes used according to the present invention is preferably carried out at pH<7 especially at pH 6.5 to pH 3.

The dyes used according to the present invention, as well as for high perspiration-, light- and colorfastness, are notable in particular for an improved migrationfastness; that is, marking off on or staining of contact material takes place even at high moisture and high temperatures, ie above 50° C., for example 60 to 100° C., to a very much smaller extent than in the case of dyes comprising a fraction of lithium ions which is outside the realm of the present invention.

The examples which follow illustrate the invention. In what follows, the dye anions of the formula I are indicated in the form of the free acid. The indicated ratios of lithium and sodium relate to the molar ratio in the dye as determined by elementel analysis for example.

### I. PREPARATION EXAMPLES

### Example 1

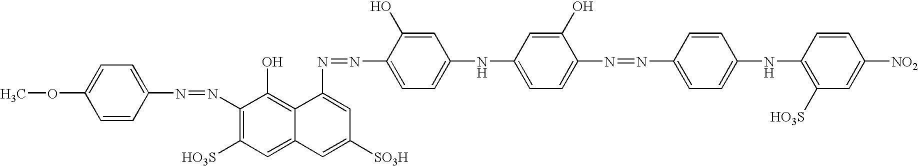

1) 1 mol of H acid (8-amino-3,6-bis(hydroxysulfonyl)-α-naphthol was dissolved in 25% by weight aqueous sodium hydroxide solution and diazotized with excess sodium nitrite and hydrochloric acid at pH<1 and 0-5° C. 0.9-1.1 mol of 3,3′-dihydroxydiphenylamine was dissolved in 50% by weight aqueous sodium hydroxide solution at pH>12 and this solution was admixed with the diazotized H acid at below 10° C. by dropwise addition over 30 minutes, during which the pH was maintained above 9 by addition of aqueous lithium hydroxide (10% by weight). After coupling was complete, the pH was adjusted to 5.5-6.5 by addition of hydrochloric acid (21% by weight).

2) 0.9-1.1 mol of 2-(4-aminophenyl)amino-5-nitrobenzenesulfonic acid was suspended in water, diazotized with excess sodium nitrite and hydrochloric acid pH<1 and 0-5° C. and the reaction mixture thus obtained was subsequently added to the reaction mixture obtained in step 1). The pH was maintained in the range pH 5.5-6.5 by addition of aqueous lithium hydroxide (10% by weight). The temperature was 15-25° C. during the coupling reaction.

3) 1 mol of p-anisidine was diazotized with excess sodium nitrite and hydrochloric acid at pH<1 and 0-5° C. and added dropwise to the reaction mixture obtained in step 2) while maintaining the pH between 5.5-6.5 with aqueous lithium hydroxide (10% by weight). The temperature of the reaction mixture was 20-25° C. After coupling, the reaction mixture was evaporated to obtain the dye.

Li/Na ratio in dye: 58/42.

### Example 2

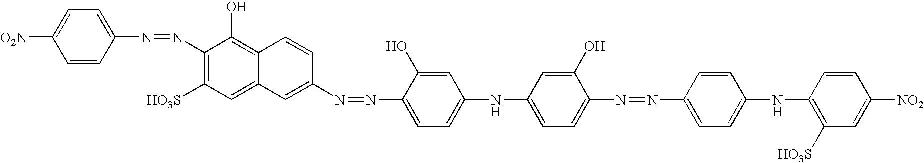

1) 1 mol of J acid was dissolved in 25% by weight aqueous sodium hydroxide solution and diazotized with excess sodium nitrite and hydrochloric acid at pH<1 and 0-5° C. 0.9-1.1 mol of 3,3′-dihydroxydiphenylamine was dissolved in 50% by weight aqueous sodium hydroxide solution at pH>12 and this solution was admixed with the diazotized J acid at below 10° C. by dropwise addition over 30 minutes, during which the pH was maintained above 9 by addition of aqueous lithium hydroxide. After coupling was complete, the pH was adjusted to 5.5-6.5 by addition of hydrochloric acid (21%).

2) 0.9-1.1 mol of 2-(4-aminophenyl)amino-5-nitrobenzenesulfonic acid was suspended in water, diazotized with excess sodium nitrite and hydrochloric acid pH<1 and 0-5° C. and the reaction mixture thus obtained was subsequently added to the reaction mixture obtained in step 1). The pH was maintained in the range pH 5.5-6.5 by addition of aqueous lithium hydroxide (10% by weight). The temperature was 15-25° C. during the coupling reaction.

3) 1 mol of p-nitroaniline was dissolved in hydrochloric acid (21% by weight) and then diazotized with excess sodium nitrite and hydrochloric acid at pH<1 and 0-5° C. and added dropwise to the reaction mixture obtained in step 2) while maintaining the pH between 5.5-6.5 with lithium hydroxide (10%). The temperature of the reaction mixture was 20-25° C. After coupling, the reaction mixture was evaporated to obtain the dye. Li/Na ratio in dye: 57/43.

### Example 3

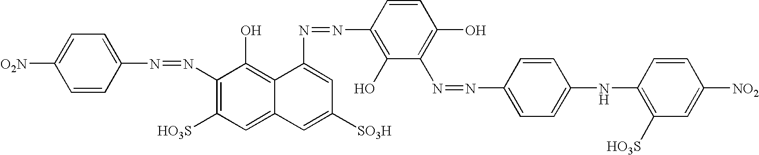

1) 1 mol of H acid was dissolved in 25% by weight aqueous sodium hydroxide solution and diazotized with excess sodium nitrite and hydrochloric acid at pH<1 and 0-5° C. 0.9-1.1 mol of resorcinol was dissolved in 50% by weight aqueous sodium hydroxide solution at pH>12 and this solution was admixed with the diazotized H acid at below 10° C. by dropwise addition over 30 minutes, during which the pH was maintained above 9 by addition of aqueous lithium hydroxide (10% by weight). After coupling was complete, the pH was adjusted to 4.5-5.5 by addition of hydrochloric acid (21% by weight).

2) 0.9-1.1 mol of 2-(4-aminophenyl)amino-5-nitrobenzenesulfonic acid was suspended in water, diazotized with excess sodium nitrite and hydrochloric acid pH<1 and 0-5° C. and the reaction mixture thus obtained was subsequently added to the reaction mixture obtained in step 1). The pH was maintained in the range pH 4.0-5.5 by addition of aqueous lithium hydroxide (10% by weight). The temperature was 15-25° C. during the coupling reaction.

3) 0.9-1.1 mol of p-nitroaniline was dissolved in hydrochloric acid (21% by weight) and then diazotized with excess sodium nitrite at pH<1 and 0-5° C. and added dropwise to the reaction mixture obtained in step 2) while maintaining the pH between 5.5-6.5 with lithium hydroxide (10%). The temperature of the reaction mixture was 20-25° C. After coupling, the reaction mixture was evaporated to obtain the dye.

Li/Na ratio in dye: 50/50.

### Example 4

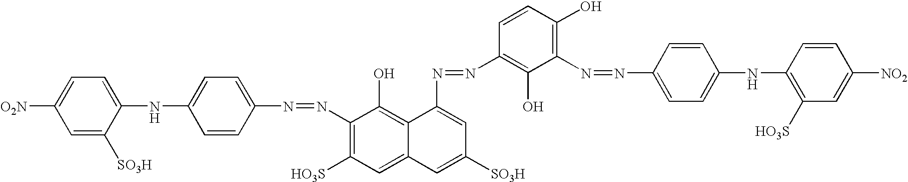

1) 1 mol of H acid was dissolved in 25% by weight aqueous sodium hydroxide solution and diazotized with excess sodium nitrite and hydrochloric acid at pH<1 and 0-5° C. 0.9-1.1 mol of resorcinol was dissolved in 50% by weight aqueous sodium hydroxide solution at pH>12 and this solution was admixed with the diazotized H acid at below 10° C. by dropwise addition over 30 minutes, during which the pH was maintained above 9 by addition of aqueous lithium hydroxide (10% by weight). After coupling was complete, the pH was adjusted to 5.5-6.5 by addition of hydrochloric acid (21% by weight).

2) 1.9-2.1 mol of 2-(4-aminophenyl)amino-5-nitrobenzenesulfonic acid was suspended in water, diazotized with excess sodium nitrite and hydrochloric acid pH<1 and 0-5° C. and the reaction mixture thus obtained was subsequently added to the reaction mixture obtained in step 1). The pH was maintained in the range 5.5-6.5 by addition of aqueous lithium hydroxide (10% by weight). The temperature was 15-25° C. during the coupling reaction. After coupling, the reaction mixture was evaporated to obtain the dye.

Li/Na ratio in dye: 38/62.

### Example 5

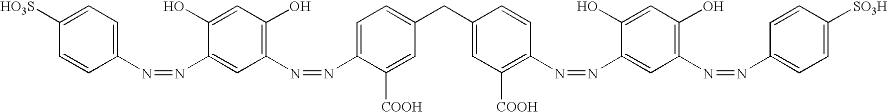

1) 1 mol of anthranilic acid was dissolved in sulfuric acid (96%) at 70-80° C. and reacted with 0.6 mol of formaldehyde. After 2 hours at 70-80° C., the reaction mixture was cooled to about 0° C. by addition of ice and the product obtained was tetrazotized with excess sodium nitrite at pH<1 and 0-5° C. 0.9-1.1 mol of resorcinol was dissolved in 50% by weight aqueous sodium hydroxide solution at pH>12 and this solution was admixed with the tetrazotized compound at below 5° C. by dropwise addition over 30 minutes, during which the pH was maintained above 8.5 by addition of aqueous hydrochloric acid (21 % by weight).

2) 1 mol of sulfanilic acid was diazotized with excess sodium nitrite and hydrochloric acid pH<1 and 0-5° C. and added dropwise to the reaction mixture obtained in step 1). The pH was maintained above 8.5 by addition of aqueous lithium hydroxide (10% by weight) while maintaining a temperature of 5-10° C. After the reaction had ended, the pH was adjusted to 4.0-5.0 with hydrochloric acid (21% by weight) and the dye was evaporated.

Li/Na ratio in dye: 55/45.

### Example 6

### 2:1 Copper Complex of the Dye Anion Depicted as a Free Acid in Example 5

1) 1 mol of anthranilic acid was dissolved in sulfuric acid (96%) at 70-80° C. and reacted with 0.6 mol of formaldehyde. After 2 hours at 70-80° C., the reaction mixture was cooled to about 0° C. by addition of ice and the product obtained was tetrazotized with excess sodium nitrite at pH<1 and 0-5° C. 0.9-1.1 mol of resorcinol was dissolved in 50% by weight aqueous sodium hydroxide solution at pH>12 and this solution was admixed with the tetrazotized compound at below 5° C. by dropwise addition over 30 minutes, during which the pH was maintained above 8.5 by addition of aqueous lithium hydroxide (10% by weight).

2) 1 mol of sulfanilic acid was diazotized with excess sodium nitrite and hydrochloric acid pH<1 and 0-5° C. and added dropwise to the reaction mixture obtained in step 1). The pH was maintained above 8.5 by addition of aqueous lithium hydroxide (10% by weight) while maintaining a temperature of 5-10° C. After the reaction had ended, the reaction solution was warmed to 80° C. and admixed with 2 mol of CuSO₄. The complexed dye was then evaporated.

LiNa ratio in dye: 55/45.

### Example 7

### 2:1 Copper Complex of the Dye Anion Depicted Hereinbelow as a Free Acid

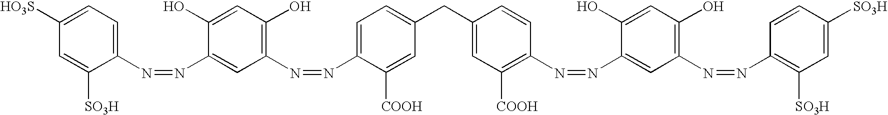

1) 1 mol of anthranilic acid was dissolved in sulfuric acid (96%) at 70-80° C. and reacted with 0.6 mol of formaldehyde. After 2 hours at 70-80° C., the reaction mixture was cooled to about 0° C. by addition of ice and the product obtained was tetrazotized with excess sodium nitrite at pH.<1 and 0-5° C. 0.9-1.1 mol of resorcinol was dissolved in 50% by weight aqueous sodium hydroxide solution at pH>12 and this solution was admixed with the tetrazotized compound at below 5° C. by dropwise addition over 30 minutes, during which the pH was maintained above 8.5 by addition of aqueous lithium hydroxide (10% by weight).

2) 1 mol of disulfanilic acid was diazotized with excess sodium nitrite and hydrochloric acid pH<1 and 0-5° C. and added dropwise to the reaction mixture obtained in step 1). The pH was maintained above 8.5 by addition of aqueous lithium hydroxide (10% by weight) while maintaining a temperature of 5-10° C. After the reaction had ended, the reaction solution was warmed to 80° C. and admixed with 2 mol of CuSO₄. Subsequently, the reaction mixture obtained was evaporated to obtain the complexed dye.

Li/Na ratio in dye: 55/45.

### Example 8

1:1 Iron Complex of the Dye Anion Depicted Hereinbelow as a Free Acid

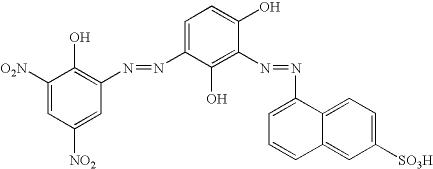

1) 1 mol of sodium picramate (6-amino-2,4-dintriophenol sodium salt) was diazotized with excess sodium nitrite and hydrochloric acid at pH<1 and 0-5° C. 0.9-1.1 mol of resorcinol was dissolved in 50% by weight aqueous sodium hydroxide solution at pH>12 and this solution was admixed with the diazotized sodium picramate at below 10° C. by dropwise addition over 30 minutes, during which the pH was maintained above 9 by addition of aqueous lithium hydroxide (10% by weight). After coupling was complete, the pH was adjusted to 4.5-5.5 by addition of hydrochloric acid (21 % by weight).

2) 0.9-1.1 mol of Cleve's acid 6 (1-aminonaphthalene-6-sulfonic acid) was suspended in water, diazotized with excess sodium nitrite and hydrochloric acid pH<1 and 0-5° C. and added dropwise to the reaction mixture obtained in step 1). The pH was maintained between 4.0 and 5.5 by addition of aqueous lithium hydroxide (10% by weight). The temperature was 15-25° C. during the coupling reaction. After the reaction had ended, the reaction solution was warmed to 60° C. and admixed with 1 mol of FeCl₃. Thereafter, the pH was raised to 6.0-6.5 by addition of aqueous sodium hydroxide solution (25%). The complexed dye was evaporated.

Li/Na ratio in dye: 28/72.

### Example 9

### 1:1 Copper Complex of the Dye Anion Depicted Hereinbelow as a Free Acid

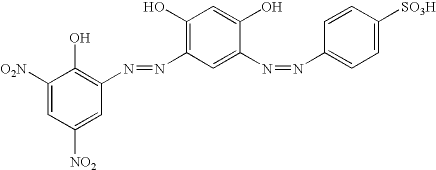

1) 1 mol of sulfanilic acid was diazotized with excess sodium nitrite and hydrochloric acid pH<1 and 0-5° C. 0.9-1.1 mol of resorcinol was dissolved in 50% by weight aqueous sodium hydroxide solution at pH>12 and admixed with the diazotized sulfanilic acid at below 10° C. by dropwise addition over 30 minutes, during which the pH was maintained above 9 by addition of aqueous lithium hydroxide (10% by weight). After the reaction was complete, the pH of the reaction mixture was adjusted to pH 7.0-8.0 with aqueous hydrochloric acid (21% by weight).

2) 0.9-1.1 mol of sodium picramate (6-amino-2,4-dinitrophenol sodium salt) was diazotized with excess sodium nitrite and hydrochloric acid pH<1 and 0-5° C. and subsequently added to the reaction mixture obtained in step 1). The pH of the reaction mixture was maintained between 7.0-8.0 by addition of lithium hydroxide (10% by weight). The temperature was maintained at 15-25° C. during the reaction. After the reaction had ended, the reaction solution was warmed to 60° C. and the pH was adjusted to 6.0-6.5 by addition of hydrochloric acid (21% by weight). 1 mol of CuSO₄ was then added. Subsequently, the reaction mixture obtained was evaporated to obtain the complexed dye.

Li/Na ratio in dye: 25/75.

### Example 10

### 1:2 Copper Complex of the Dye Anion Depicted Hereinbelow as a Free Acid

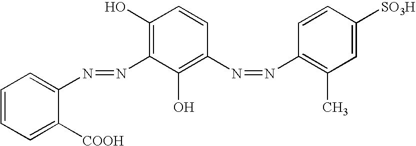

1) 1 mol of o-toluidine acid (4-amino-3-methylbenzenesulfonic acid) was diazotized with excess sodium nitrite and hydrochloric acid pH<1 and 0-5° C. 0.9-1.1 mol of resorcinol was dissolved in 50% by weight aqueous sodium hydroxide solution at pH>12 and admixed with the diazotized o-toluidine acid at below 10° C. by dropwise addition over 30 minutes, during which the pH was maintained above 9 by addition of aqueous lithium hydroxide (10% by weight). After the reaction was complete, the pH of the reaction mixture was adjusted to 4.5-5.5 with hydrochloric acid (21% by weight).

2) 0.9-1.1 mol of anthranilic acid was diazotized with excess sodium nitrite and hydrochloric acid pH<1 and 0-5° C. and subsequently added to the reaction mixture obtained in step 1). The pH of the reaction mixture was maintained between 4.0 and 5.5 by addition of lithium hydroxide (10% by weight). The temperature was maintained at 15-25° C. during the reaction. After the reaction had ended, the reaction solution was warmed to 60° C. and the pH was adjusted to 6.0-6.5 by addition of hydrochloric acid (25% by weight). 0.5 mol of CuSO₄ was then added. Subsequently, the reaction mixture obtained was evaporated to obtain the complexed dye.

Li/Na ratio in dye: 33/67.

### Example 11

### 1:2 Copper Complex of the Dye Anion Depicted Hereinbelow as a Free Acid

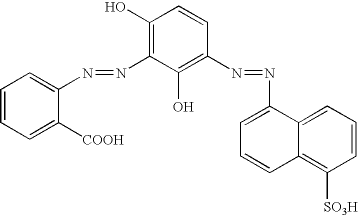

1) 1 mol of purpurin acid (1-aminonaphthalene-5-sulfonic acid) was diazotized with excess sodium nitrite and hydrochloric acid pH<1 and 0-5° C. 0.9-1.1 mol of resorcinol was dissolved in 50% by weight aqueous sodium hydroxide solution at pH>12 and admixed with the diazotized purpurin acid at below 10° C. by dropwise addition over 30 minutes, during which the pH was maintained above 9 by addition of aqueous lithium hydroxide (10% by weight). After the reaction was complete, the pH of the reaction mixture was adjusted to 4.5-5.5 with hydrochloric acid (21% by weight).

2) 0.9-1.1 mol of anthranilic acid was diazotized with excess sodium nitrite and hydrochloric acid pH<1 and 0-5° C. and subsequently added to the reaction mixture obtained in step 1). The pH of the reaction mixture was maintained between 4.0 and 5.5 by addition of lithium hydroxide (10% by weight). The temperature was maintained at 15-25° C. during the reaction. After the reaction had ended, the reaction solution was warmed to 60° C. and the pH was adjusted to 6.0-6.5 by addition of aqueous sodium hydroxide solution (25% by weight). 0.5 mol of CuSO₄ was then added. Subsequently, the reaction mixture obtained was evaporated to obtain the complexed dye.

Li/Na ratio in dye: 31/69.

### Example 12

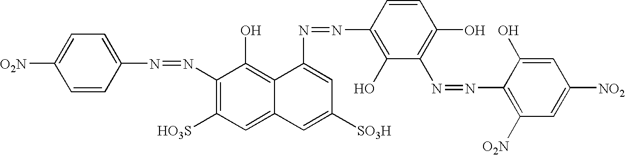

1) 1 mol of H acid was dissolved in 25% by weight aqueous sodium hydroxide solution and diazotized with excess sodium nitrite and hydrochloric acid at pH<1 and 0-5° C. 0.9-1.1 mol of resorcinol was dissolved in 50% by weight aqueous sodium hydroxide solution at pH>12 and this solution was admixed with the diazotized H acid at below 10° C. by dropwise addition over 30 minutes, during which the pH was maintained above 9 by addition of aqueous lithium hydroxide (10% by weight). After coupling was complete, the pH was adjusted to 4.5-5.5 by addition of hydrochloric acid (21% by weight).

2) 0.9-1.1 mol of sodium picramate was suspended in water, diazotized with excess sodium nitrite and hydrochloric acid pH<1 and 0-5° C. and the reaction mixture thus obtained was subsequently added to the reaction mixture obtained in step 1). The pH of the reaction mixture was maintained in the range between 4.0 and 5.5 by addition of lithium hydroxide (10% by weight). The temperature was 15-25° C. during the reaction.

3) 0.9-1.1 mol of p-nitroaniline was dissolved in hydrochloric acid (21%) and then diazotized with excess sodium nitrite at pH<1 and 0-5° C. and then added dropwise to the reaction mixture obtained in step 2). The pH of the reaction mixture was maintained between 4.0 and 5.5 by the addition of lithium hydroxide (10% by weight). The temperature during the reaction was 20-25° C. Subsequently, the reaction mixture obtained was evaporated to obtain the complexed dye.

Li/Na ratio in dye: 60/40.

### Example 13

### 1:1 Iron Complex of the Dye Anion Depicted hereinbelow as a Free Acid

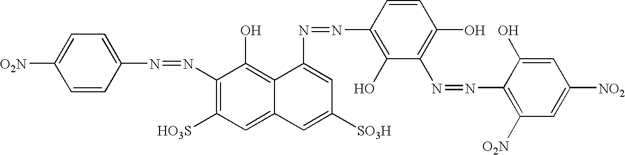

1) 1 mol of H acid was dissolved in 25% by weight aqueous sodium hydroxide solution and diazotized with excess sodium nitrite and hydrochloric acid at pH<1 and 0-5° C. 0.9-1.1 mol of resorcinol was dissolved in 50% by weight aqueous sodium hydroxide solution at pH>12 and this solution was admixed with the diazotized H acid at below 10° C. by dropwise addition over 30 minutes, during which the pH was maintained above 9 by addition of aqueous lithium hydroxide (10% by weight). After coupling was complete, the pH was adjusted to 4.5-5.5 by addition of hydrochloric acid (21% by weight).

2) 0.9-1.1 mol of sodium picramate was suspended in water, diazotized with excess sodium nitrite and hydrochloric acid pH<1 and 0-5° C. and the reaction mixture thus obtained was subsequently added to the reaction mixture obtained in step 1). The pH of the reaction mixture was maintained between 4.0 and 5.5 by addition of aqueous lithium hydroxide (10% by weight). The temperature was 15-25° C. during the reaction.

3) 0.9-1.1 mol of p-nitroaniline was dissolved in hydrochloric acid (21%) and then diazotized with excess sodium nitrite at pH<1 and 0-5° C. and then added dropwise to the reaction mixture obtained in step 2). The pH of the reaction mixture was maintained between 4.0 and 5.5 by the addition of lithium hydroxide (10% by weight). The temperature during the reaction was 20-25° C. The reaction mixture obtained was then warmed to 60° C. and adjusted to pH 6.0-6.5 by addition of aqueous sodium hydroxide solution (25% by weight). Thereafter, 1 mol of FeCl₃ was added. The reaction mixture obtained was then evaporated to obtain the complexed dye.

Li/Na ratio in dye: 54/66.

### Example 14

### 1:1 Iron Complex of the Dye Anion Depicted Hereinbelow as a Free Acid

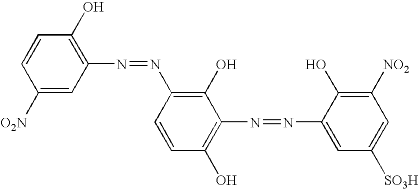

1) 1 mol of 2-amino-4-nitrophenol was diazotized with excess sodium nitrite and hydrochloric acid pH<1 and 0-5° C. 0.9-1.1 mol of resorcinol was dissolved in 50% by weight aqueous sodium hydroxide solution at pH>12 and admixed with the diazotized 2-amino-4-nitrophenol at below 10° C. by dropwise addition over 30 minutes, during which the pH was maintained above 9 by addition of aqueous lithium hydroxide (10% by weight). After reaction was complete, the pH of the reaction mixture was adjusted to 4.5-5.5 with hydrochloric acid (21% by weight).

2) 0.9-1.1 mol of 4-nitro-2-aminophenol-6-sulfonic acid was diazotized with excess sodium nitrite and hydrochloric acid at pH<1 and 0-5° C. and then added to the reaction mixture obtained in step 1). The pH of the reaction mixture was maintained between 4.0 and 5.5 in the process by addition of lithium hydroxide (10% by weight). The temperature was maintained at 15-25° C. during the reaction. After the reaction had ended, the reaction solution was heated to 60° C. and adjusted to pH 6.0-6.5 by addition of aqueous sodium hydroxide solution (25% by weight). 1 mol of FeCl₃ was then added. The reaction mixture obtained was then evaporated to obtain the complexed dye.

Li/Na ratio in dye: 41/59.

### Example 15

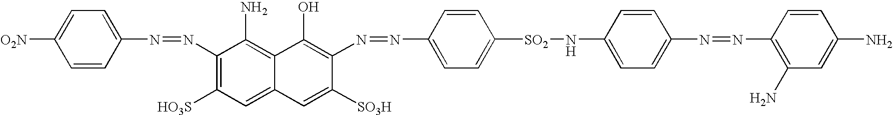

1) 1 mol of p-nitroaniline was dissolved in hydrochloric acid (21% by weight) and thereafter diazotized with excess sodium nitrite at pH<1 and 0-5° C. 1 mol of H acid was added, while the pH was maintained below 1.5. Subsequently the reaction mixture was adjusted to pH 9-10 by addition of aqueous lithium hydroxide (10% by weight).

2) 1 mol of 4,4-diaminodiphenylsulfamide was tetrazotized with excess sodium nitrite and hydrochloric acid at pH<1 and 0-5° C. and added to the reaction mixture obtained in step 1) while maintaining a pH of 9-10 by addition of aqueous lithium hydroxide (10% by weight).

3) After the reaction in step 2) was complete, 1 mol of m-phenylenediamine was added in one portion into the reaction mixture obtained in step 2) while the pH was maintained between 9-10 by addition of aqueous lithium hydroxide (10% by weight). The temperature during the reaction was maintained in the range of 20-30° C. The reaction mixture was then evaporated to obtain the dye.

Li/Na ratio in dye: 45/55.

### Example 16

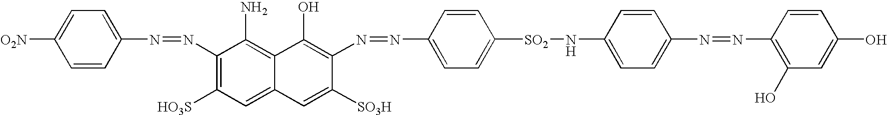

1) 1 mol of p-nitroaniline was dissolved in hydrochloric acid (21% by weight) and thereafter diazotized with excess sodium nitrite at pH<1 and 0-5° C. 1 mol of H acid was added, while the pH was maintained below 1.5. Subsequently the reaction mixture was adjusted to pH 9-10 by addition of aqueous lithium hydroxide (10% by weight).

2) 1 mol of 4,4-diaminodiphenylsulfamide was tetrazotized with excess sodium nitrite and hydrochloric acid at pH<1 and 0-5° C. and added to the reaction mixture obtained in step 1) while maintaining a pH of 9-10 by addition of aqueous lithium hydroxide (10% by weight).

3) After the reaction in step 2) was complete, 1 mol of resorcinol was added in one portion into the reaction mixture obtained in step 2) while the pH was maintained between 9-10 by addition of aqueous lithium hydroxide (10% by weight). The temperature during the reaction was maintained in the range of 20-30° C. The reaction mixture was then evaporated to obtain the dye.

Li/Na ratio in dye: 43/57.

### II. Performance Testing

1. Leather Dyeing (General Prescription)

A piece of 100 parts by weight of a conventionally chrome-tanned cattlehide leather 1.1 mm in shaved thickness was washed at 40° C. for 10 min in drums filled with 300 parts of water and subsequently neutralized at 35° C. in a liquor consisting of 150 parts of water, 1.5 parts of sodium formate and 1.5 parts of sodium bicarbonate (dilution 1:15), 2 parts of a polymeric retanning agent and 2 parts of an anionic fatliquor (dilution 1:3) for 90 min. Thereafter, the deacidifying liquor had a pH of 6.3. This was followed by a 10 minute wash in 200 parts of water at 40° C. The leather thus treated was retanned at 40° C. for 20 min in a freshly prepared liquor composed of 100 parts of water and 3 parts of a polymeric retanning material (dilution 1:3). The liquor thereafter had a pH of 6.0. Subsequently, the liquor was admixed with 4 parts of a natural-based fatliquor and after 30 min with 3 parts of a synthetic retanning agent and 4 parts of tara (vegetable tanning material). After 30 min, the liquor was admixed with 4.0 parts of the inventive or comparative dyeing agent. After 60 min, 100 parts of water were added and the leather was drummed at 70° C. for 20 min. The leather was subsequently fatliquored in the same liquor with 5 parts of a natural-based fatliquor and 3 parts of a polymeric fatliquor (dilution 1:3) at 50° C. by drumming for one hour. Finally, the leather was acidified with 2 parts of a concentrated hydroformic acid to pH 3.6 and drummed twice for 20 min and once for 40 min. The dyed and fatliquored leather was additionally rinsed for 10 min with cold water at 15° C. and finally set out, suspension dried at room temperature overnight, sawdusted, staked and strained.

Leather was dyed with the dyes of preparation examples 1 to 16 by the general dyeing prescription described above. For comparison, the leather was dyed with dyes corresponding to those of examples 1 to 16 except that they comprised no lithium.

The migrationfastness level of the leather pieces thus dyed against contact material such as, for example PVC and crepe rubber was determined under high temperatures (e.g. 60, 70, 80 or 90° C.) and also under high humidity.

Furthermore, the migration into white-pigmented dressing layers based on polyacrylate, polyurethane or polybutadiene was determined under high humidity and high temperatures.

The differences in the migrationfastness level between the corresponding dyes was rated by visual inspection. It was found that all the dyes which comprised lithium were superior in migrationfastness to the corresponding, sodium-synthesized dyes by a half to a whole point (assessment against ISO 105-A03 gray scale, 1 severe staining, 5 no staining).

1-12. (canceled)

13. A process for dyeing leather, comprising treating leather with an aqueous solution of a dye wherein the dye comprises at least one anionic azo dye or a transition metal complex thereof, the anionic azo dye having at least one acidic group in the form of its salt and at least 2 azo groups and wherein dye anion is described by the general formula I 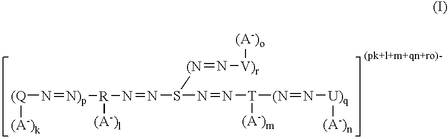
where
Q, R, T, U and V are independently a benzene, naphthalene, diphenylamine, diphenylmethane, phenylaminosulfonylbenzene or phenylaminocarbonyl-benzene radical and where Q and U may also be a pyrazole, thiazole, triazole, isothiazole or thiadiazole radical and as well as by the optionally present anionic group A⁻ may optionally be substituted by one or more of the following radicals: OH, NH₂, NO₂, halogen, C₁-C₄-alkyl, C₁-C₄-hydroxyalkyl, C₁-C₄-alkoxy, C₁-C₄-alkylamino, C₁-C₄-dialkylamino, C₁-C₄-alkylaminocarbonyl,
C₁-C₄-dialkylaminocarbonyl, C₁-C₄-alkylcarbonylamino,
N-(C₁-C₄-alkylcarbonyl)-N-(C₁-C₄-alkylcarbonyl)amino, phenylcarbonylamino, formamide or N-attached 5- or 6-membered heterocyclyl, where Q and U may independently have an —N═N-P substituent, where P is an aromatic radical which is selected from the group consisting of phenyl, naphthyl, pyridyl, 2-pyridonyl, pyrazolyl and thiazolyl and wherein P is optionally substituted by one or more radicals selected from the group consisting of OH, NH₂, NO₂, halogen, C₁-C₄-alkyl, C₁-C₄-hydroxyalkyl, C₁-C₄-alkoxy, C₁-C₄-alkylamino, C₁-C₄-dialkylamino, C₁-C₄-alkylaminocarbonyl, C₁-C₄-dialkylaminocarbonyl, C₁-C₄-alkylcarbonylamino, N-(C₁-C₄-alkylcarbonyl)-N-(C₁-C₄-alkylcarbonyl)amino, phenylcarbonylamino, formamide, N-attached 5- or 6-membered saturated nitrogen heterocyclyl, morpholinyl, piperazinyl and pyrrolidinyl;
S is a radical of the formulae A or B; 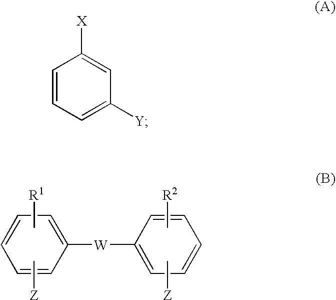
where X and Y in the formula A are independently OH, NH₂, C₁-C₄-alkyl, C₁-C₄-hydroxyalkyl, C₁-C₄-alkoxy, C₁-C₄-alkylamino, C₁-C₄-dialkylamino or N-attached 5- or 6-membered saturated heterocyclyl, W in the formula B is a bond, O, NH, SO₂, SO₂NH, CONH or C₁-C₄-alkylene, R¹ and R² are independently hydrogen, OH, or C₁-C₄-alkyl and each Z is the same or different and represents COOH or hydrogen;
A⁻ may at each instance be the same or different and is selected from the group consisting of CO₂⁻ and SO₃⁻;
k, l, m, n and o may be the same or different and are each 0, 1 or 2, provided the sum total pk+l+m+qn+ro is at least 1;
p, q and r are independently 0 or 1;
which comprise lithium ions and sodium ions in a molar ratio in the range from 1:19 to 4:1 and especially in a molar ratio in the range from 1:9 to 3.5:1.

14. The process as claimed in claim 13, wherein the sum total pk+l+m+qn+ro is 2, 3, 4, 5 or 6.

15. The process as claimed in claim 13, wherein the total amount of lithium ions and sodium ions accounts for at least 90% and at least 95% of the cations required to neutralize the anionic groups in the dye.

16. The process as claimed in claim 13, wherein S in the formula I is a radical of formula A.

17. The process as claimed in claim 13, wherein the sum total p+q+r in the formula I is 1 or 2.

18. The process as claimed in claim 13, wherein at least one of the groups Q-(A⁻)ₖ, R-(A⁻)ₗ, T-(A⁻)ₘ, U-(A⁻)ₙ, or V-(A⁻)ₒ, is described by the general formula II: 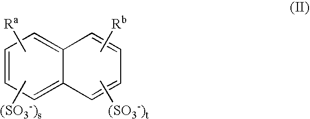
where R^{a}, R^{b}, are independently hydrogen, OH, NH₂ or NHC(O)R³, where R³ is hydrogen, C₁-C₄-alkyl or phenyl, s and t are each 0 or 1 and the sum total s+t is 1 or 2.

19. The process as claimed in claim 18, wherein the dye anion has the formula Ia: 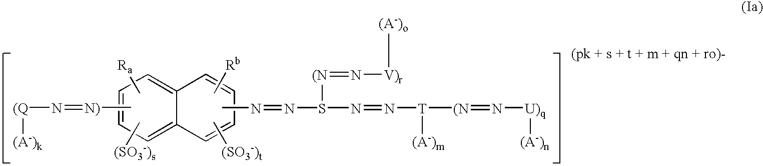
where R^{a}, R^{b}, A, S, T, U, V, k, m, n, p, q, o, r are each as defined above, a and b are independently 0 or 1, s and t are independently 0 or 1 and the sum total s+t is 1 or 2.

20. The process as claimed in claim 18, wherein the anions I have the following formulae Ia.1, Ia.2, Ia.3, Ia.4, Ia.5, Ia.6, Ia.7 or Ia.8: 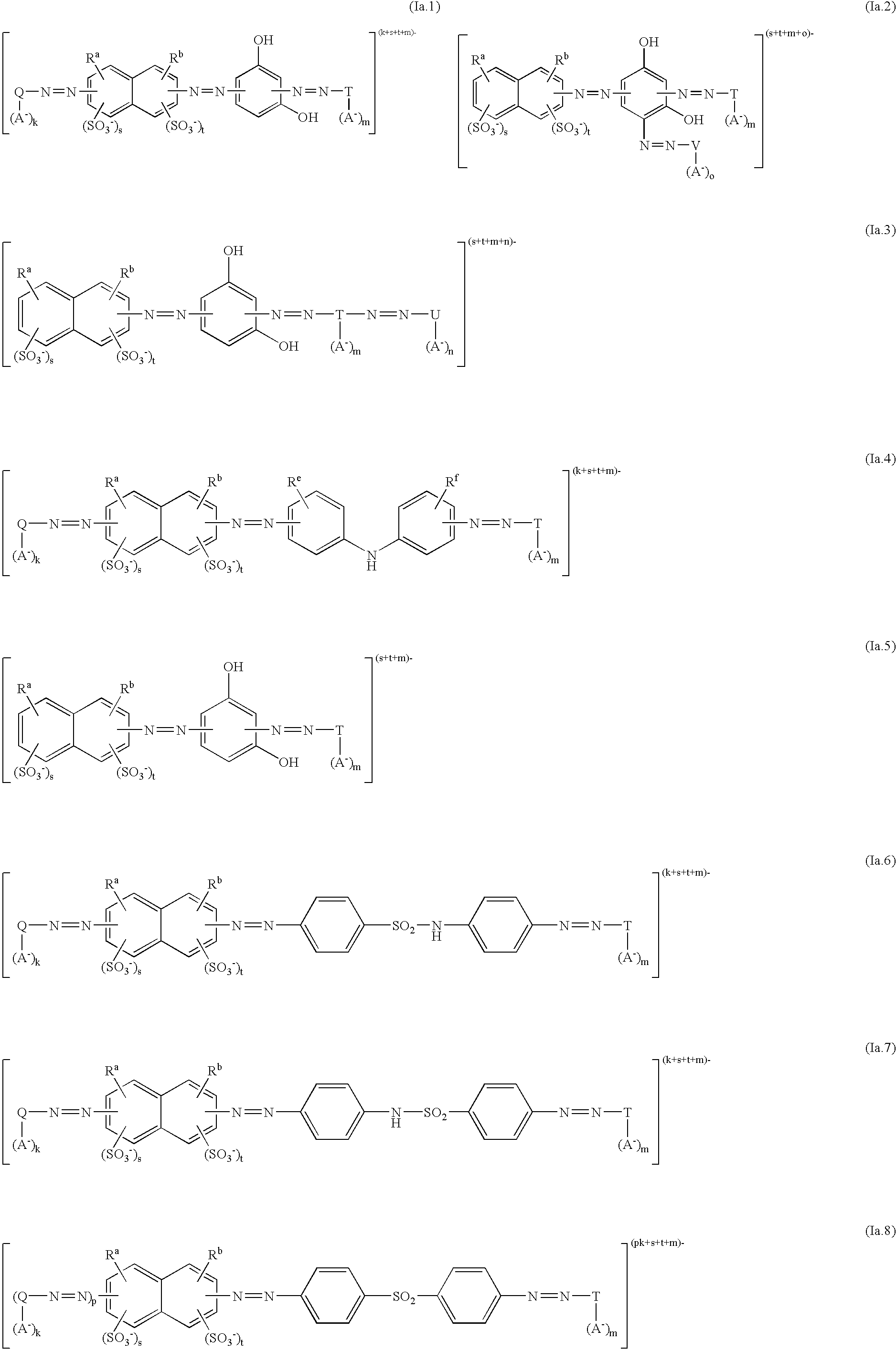
where R^{a}, R^{b}, A, T, U, V, k, m, n, p, are each as defined above, s and t are independently 0 or 1, the sum total s+t is 1 or 2 and R^{e} and R^{f} are independently H, C₁-C₄-alkyl or OH.

21. The process as claimed in claim 13, wherein the anions have the following formula Ib: 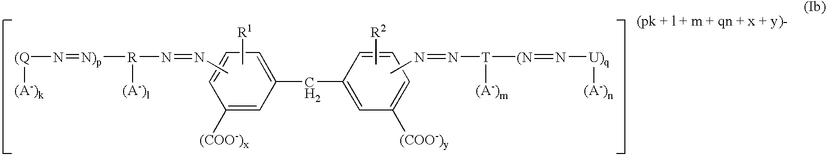
where A⁻, Q, R, R¹, R², T, U, k, l, m, n, p and q are each as defined above and x and y are independently 0 or 1.

22. The process as claimed in claim 13, wherein the anion I has the following formula Ic: 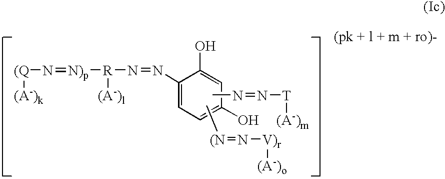
where A⁻, k, l, m, o, p and r are each as defined above, R and T are independently a benzene or diphenylamine radical, V and Q are independently a benzene radical and R, T, Q and V as well as the anionic group A⁻ optionally present may optionally be substituted by one or more of the following radicals: OH, NH₂ NO₂, halogen, C₁-C₄-alkyl, C₁-C₄-hydroxyalkyl, C₁-C₄-alkoxy, C₁-C₄-alkylamino, C₁-C₄-dialkylamino, C₁-C₄-alkylaminocarbonyl, C₁-C₄-dialkylaminocarbonyl, C₁-C₄-alkylcarbonylamino, N-(C₁-C₄-alkylcarbonyl)-N-(C₁-C₄-alkylcarbonyl)amino.

23. The process as claimed in claim 13, wherein the anionic azo dyes and of their anionic metal complexes which have at least one acidic group in the form of their salt and at least 2 azo groups and whose dye anion is described by the general formula I, wherein the dye anion of the general formula I is obtained by a process which comprises at least two successive azo couplings which are carried out in an aqueous reaction medium which comprises at least 90 mol %, based on the metal ions present in the reaction medium, of sodium ions and lithium ions in a Na:Li molar ratio in the range from 95:5 to 20:80.
